(19) United States
(12) Patent Application Publication
(10) Pub. No.: US 2003/0003523 A1
(43) Pub. Date: Jan. 2, 2003

(54) Conductometric detection process

(76) Inventor: Mark Jensen, West Chester, PA (US)

Correspondence Address: Bennett J. Berson, Quarles & Brady LLP, 1 South Pinckney Street, P O Box 2113, Madison, WI, 53701-2113, US

(21) Appl. No.: 10/174,869

(22) Filed: Jun. 18, 2002

Related U.S. Application Data
(60) Provisional application No. 60/299,278, filed on Jun. 19, 2001.

(51) Int. Cl. C12Q 1/26; C25D 3/46; C12M 1/34

(52) U.S. Cl. 435/25; 435/287.1; 205/777.5; 205/263

(57) ABSTRACT

The present invention provides a method for determining in a sample the presence and the level of a substance to be detected that can specifically bind to a capture substance. The method involves providing complexes comprising the substance to be detected, the capture substance and an oxidative enzyme linked to the complexes on a base along a path defined by a pair of electrical conductors. The oxidative enzyme promotes amplifying deposition of a metal layer along the path such that the metal layer becomes more electrically conductive, thereby enhancing the detection sensitivity. One can determine the presence and the level of the detected substance by monitoring the current flow of an electric circuit that includes the path.

### CROSS-REFERENCE TO RELATED APPLICATION

This application claims the benefit of US provisional application Ser. No. 60/299,278, filed on Jun. 19, 2001, which is incorporated herein by reference as if set forth in its entirety.

### STATEMENT REGARDING FEDERALLY SPONSORED RESEARCH OR DEVELOPMENT

Not applicable.

### BACKGROUND OF THE INVENTION

U.S. Pat. Nos. 4,794,089, 5,137,827 and 5,284,748, each incorporated herein by reference as if set forth in its entirety, typify methods and devices for electronic detection of a binding reaction. U.S. Pat. No. 4,794,089 teaches binding a substance to be detected in a sample (the “detected substance”) to electrically conductive particles provided between a pair of spaced-apart electrical conductors such that a change in current flow resulting from the binding indicates the presence of the bound substance in the sample. The conductive particles can be, e.g., a colloidal gold-antibody conjugate or a colloidal gold-streptavidin conjugate. U.S. Pat. No. 5,137,827 discloses a related diagnostic element. U.S. Pat. No. 5,284,748 discloses refined methods for detecting a substance in a sample by complexing the detected substance on the surface of electrically conductive particles and then binding the complexes thus formed between a pair of spaced-apart conductors. The '748 patent further discloses a signal enhancing step of coating the bound complexes with an electrically conductive enhancer such as silver.

In the noted patents, for example, a capture substance provided between the conductors on the base defines a path between the conductors. If a tested sample contains the detected substance, aggregates containing a complex of the capture substance and the detected substance form under conditions that allow specific binding between the two. Other binding and signal-enhancement strategies are also described in the incorporated patents. In practice, signal enhancement is limited because the electrically conductive enhancer binds only to specific sites labeled with a conductive particle and each labeling event yields just one site that can be enhanced. A more substantial signal enhancement method is needed to increase the sensitivity of the method and to improve the ability to quantify the amount of a substance detected in a sample.

U.S. Pat. No. 5,116,734, incorporated herein by reference as if set forth in its entirety, discloses a sensitive method for visualizing tissues or membranes stained with a horseradish peroxidase (HRP)-labeled probe. The tissues or membranes are exposed to 3,3′-diaminobenzidine (3,3′-DAB or simply DAB) and a divalent metal cation (M²⁺) such as nickel (Ni²⁺) and, in the presence of an oxygen source such as hydrogen peroxide (H₂O₂), HRP-catalyzed oxidation precipitates a complex of oxidized DAB and reduced metal M^{o}. Subsequent steps in the method include washing at low pH to reduce background, treating the M^{o} with a gold toning solution (comprising, e.g., HAuCi₄) to plate Au^{o} onto the M^{o}, adding a silver (Ag⁺) enhancer to precipitate Ag^{o} onto the Au^{o}. The process of the patent is adjusted to optimize visual resolution and does not necessarily yield a conductive surface, even though metal is precipitated.

### BRIEF SUMMARY OF THE INVENTION

The present invention provides a method for determining in a sample the presence and level of a detected substance that can specifically bind to a capture substance that is itself bound between a pair of spaced-apart electrical conductors on a substantially electrically non-conductive base to define a path between the conductors. As in prior methods, when the detected substance present in the sample is exposed to the bound capture substance, an electrically conductive path forms on the path, in any of the various manners provided for in incorporated U.S. Pat. Nos. 4,794,089, 5,137,827 and 5,284,748. In one approach to the present invention, for example, the capture substance is bound to the base and a sample that contains the detected substance is exposed to the capture substance whereupon a complex of the two substances is formed as in the prior methods.

However, the present invention improves the detection sensitivity of prior methods by employing an amplifying oxidative chemistry for further depositing a conductive transition metal layer on the bound electrically-conductive aggregates in the path between the conductors. In the method, the aggregate formed between the capture substance and the detected substance comprises an oxidative enzyme. When the aggregates with oxidative enzyme are exposed to an oxidizable substrate and transition metal ions, the oxidative enzyme catalyzes the substrate and with a concurrent reduction of the metal ions to the zero-valence state. The oxidized substrate and the zero-valence metal atoms precipitate to form an amplified conductive transition metal layer on the path. When the electrical conductors are connected to an electrical energy source to form an electrical circuit that includes the amplified conductive layer on the path, the circuit is characterized by a lower resistance than a circuit lacking the amplified conductive layer or having fewer aggregates in the layer. Accordingly, the amount of the detected substance can be characterized by the electrical current flow relative to a control circuit in which the detected substance is absent or present in known quantity.

For still further enhanced sensitivity, a layer of metal, preferably gold, can optionally be deposited on the transition metal layer as described herein. Other suitable metals can include platinum and palladium. Additionally, if the optional metal layer is provided, a yet further enhancement can be obtained by depositing silver on the metal layer. The resulting conduction along the path thus created can reveal the presence of the detected substance, and if the assay is properly configured, the amount in the sample.

Advantageously the method of the present invention is more sensitive than prior methods, because the method exponentially amplifies each complex formed on the path between the capture substance and the detected substance into a large number of deposited transition metal atoms that can be quantified. The method can be quantitative because the conductivity (and thickness) of the transition metal layer formed in the method is directly proportional to the amount of oxidative enzyme in the aggregates and, therefore, to the amount of the detected substance in the sample.

In addition to the aforementioned sensitivity improvements, the method of the present invention is also more cost effective than the colloidal gold conjugates of the prior art systems because the oxidative enzymes such as HRP are substantially less expensive than colloidal gold.

Other objects, advantages and features of the present invention will become apparent from the following specifications and claims.

### BRIEF DESCRIPTION OF THE SEVERAL VIEWS OF THE DRAWINGS

Not applicable.

### DETAILED DESCRIPTION OF THE INVENTION

The present invention is a sensitive electronic detection method for determining the presence and the level of a detected substance that can specifically bind to a capture substance. The method of the present invention can be employed in existing detection assays such as sandwich assays for detecting a nucleic acid, polypeptide or protein.

An oxidative enzyme that promotes the deposition of a transition metal layer is linked directly or indirectly to a component of the aggregate complex that comprises the detected and the capture substances. The enzyme can be linked to the complex component before or after the complex is formed or deposited in the path on the base. An indirect enzyme linkage can be a linkage to a molecule that is, in turn, linked to either complex component. In the presence of an oxidizable substrate and ions of a transition metal, the oxidative enzyme catalyzes an oxidation reaction such that the substrate is oxidized and the metal ions are reduced to zero-valence metal atoms. The oxidized substrate and the metal atoms precipitate onto the aggregate and form a metal layer.

The following depicts the principle of the invention using sandwich hybridization assays, although other binding or hybridization strategies are readily apparent to the skilled artisan. The following also employs the preferred oxidative enzyme detection chemistry, namely HRP-based detection. In one embodiment, target nucleic acid directly labeled with biotin, such as a product of PCR amplification, is captured on a capture nucleic acid anchored to the path in the base by nucleic acid hybridization under suitable hybridization conditions reflecting the degree of complementarity between the detected nucleic acid and its capture nucleic acid. Such conditions can readily be determined by the skilled artisan. HRP-labeled streptavidin is then bound to the biotin-tagged target nucleic acid, and the HRP is detected as described below. In a second embodiment, target nucleic acid is captured on a capture nucleic acid anchored to the path in the base. A biotin-tagged nucleic acid is allowed to hybridize to the captured target nucleic acid under suitable hybridization conditions. HRP-labeled streptavidin is then bound to the biotin-tagged second nucleic acid, and the HRP is detected as described below. In a third related sandwich assay, an antibody capable of specific binding to an antigen of interest is anchored to the path in the base. The antigen of interest is then bound to the captured antigen under suitable binding conditions. An HRP-labeled second antibody capable of binding to the antigen is then bound to the antibody-antigen complex and the HRP is detected as described below. A skilled artisan understands that either antigen or antibody can be detected in this manner by anchoring either an antigen or an antibody and adjusting the reagents accordingly.

To detect the HRP in either of the above-noted sandwich complexes, DAB is added (to about 0.05-5 mg/ml), H₂O₂ is added (to about 0.05-10 mg/ml) and divalent metal is added (to about 1-30 mM). The bound HRP catalyzes the formation of sufficient O₂ from H₂O₂ to oxidize enough DAB to form a polymer that precipitates. While the DAB oxidation occurs, some oxidized DAB is further oxidized by the divalent metal ions, thereby reducing the metal ions to zero-valence metal atoms that co-precipitate with the polymerized DAB to form a metal layer that is typically less than 100 nm thick. Although the resistance of the metal layer can be measured in some cases, preferred embodiments of the invention include the following enhancement steps.

Next, optionally, a gold source such as HAuCl₄ is added (to about 1 μM -1 mM) and is exposed to the metal-DAB precipitate until gold metal is deposited on the metal surface, typically about 10 minutes. When the reaction is performed on a plastic surface, non-specific gold plating on the oxidized DAB and on the plastic can occur at higher gold levels or at appreciably longer reaction times, resulting in high background noise. A skilled artisan practicing the invention on a different solid-phase surface can choose an appropriate reaction time and amount of gold by applying routine assay design procedures to adjust the background to a level acceptable for the reaction of interest.

Finally, if the prior gold step is used, Ag⁺ is added (to about 1 mM-50 mM) and hydroquinone is added (to about 10 mM-200 mM), whereby the gold metal on the surface of the metal layer catalyzes reduction by the hydroquinone of Ag⁺ to yield silver metal and benzoquinone. The silver metal is deposited onto the gold surface to form a conductive metal surface. Once this begins, the native Ag surface also catalyzes the same reaction.

The steps in the preferred chemical process are summarized below:

Bound peroxidase+DAB+H₂O₂+M²⁺

[symbol]

DAB+O₂+M²⁺=<DAB(ox)M^{o} (i)

DAB(ox)M^{o}+HAuCl₄=<DAB(ox)M^{o}Au^{o} (ii)

DAB(ox)M^{o}Au^{o}+Ag⁺+Hydroquinone=<DAB(ox)M^{o}Au^{o}Ag^{o}+Benzoquinone (iii)

The use of an oxidative enzyme allows one binding event to cause the initial deposition in a conductive layer of a large number of transition metal atoms, which can optionally serve in turn as nucleation sites for gold deposition and silver enhancement. The result is a substantial increase in detection sensitivity. The resistance of the conductive metal layer formed is inversely proportional to the thickness of the layer. The thickness of the metal layer is determined, at least in part, by the number of aggregates formed on the path. One of ordinary skill in the art can easily adjust the method parameters so that the detected substance can be quantified. One can also use the method to quantify molecules and complexes of molecules, such as the oxidative enzyme or an enzyme conjugate in the aggregates, the amount of which also correlates with the number of the aggregates formed.

Any combination of the aforementioned oxidative enzymes, oxidizable substrates and transition metal ions or others that function as described above can be used in the present invention. Suitable oxidative enzymes for the present invention have a turnover rate that exceeds 10⁴/second and can include but are not limited to peroxidases and oxidases. A preferred oxidative enzyme for use in the method is a peroxidase, still more preferably a horseradish peroxidase (HRP). HRP has been used extensively for commercial and research purposes. Suitable oxidative substrates include but are not limited to DAB, and 3,3′,5,5′-tetramethylbenzidine. Suitable transition metal ions include but are not limited to an ion of nickel, cobalt, copper, lead, or cadmium.

As noted, the metal layer can be treated with a gold source, such as a gold salt, under conditions suitable for depositing a gold metal onto the transition metal layer, thereby providing a second metal layer that is more catalytic than the transition metal layer for the subsequent reduction of silver ions. A non-limiting example of a suitable gold source for gold treatment is HAuCl₄.

If the gold treatment is performed, a further optional step of depositing silver on the gold metal can be performed. Silver deposition is accomplished by treating the gold metal with a source of silver ion and a reducing agent. Non-limiting examples of a suitable silver ion source are AgNO₃, silver acetate or silver lactate. A non-limiting example of a suitable reducing agent is hydroquinone.

In one embodiment, the invention is a method for determining the presence and level of a detected substance in a sample where the capture substance is bound to the base. In this embodiment, one simply exposes the sample to the capture substance bound on the base under conditions that allow specific binding between the two substances followed by steps of producing a metal layer on the base and detecting the metal layer, as described above.

In another embodiment, internal controls are incorporated into the assay for determining the presence of, and, optionally, the level of, the detected substance in an original sample. In this embodiment, the detected substance is adhered to the base to define a path between the conductors. An aliquot of the original sample is then separately mixed with a known amount of the capture substance, under conditions that allow specific binding between the capture substance and the detected substance, to form a test sample.

For a quantitative assay, the known amount of capture substance mixed with the aliquot of original sample must exceed the amount that can bind up all of the detected substance such that the test sample contains free (i.e., excess) capture substance. The test sample is then exposed to the detected substance adhered on the base under conditions that permit the free capture substance to bind to the adhered detected substance. The amount of capture substance in the test sample is then determined as described herein. The amount of unbound capture substance in the test sample correlates inversely with the amount of detected substance in the original sample. A separate negative control sample lacking the substance to be detected in the original sample, is preferably processed in parallel with the test sample. A comparison of the change in electrical current flow between the negative control sample and the test sample indicates the presence and quantity of the detected substance in the original sample, whereby an increase in resistance relative to the negative control signals the presence of the detected substance in the original sample.

When the known amount of capture substance added to the aliquot of original sample is not sufficient to bind all of the detected substance, the assay can be a qualitative indicator of presence or absence of the detected substance. In this case, if the original sample contains the detected substance, no free capture substance is available to bind in the path on the base, thereby signaling by high resistance the presence of the detected substance in the original sample. Alternatively, a resistance lower than a positive control indicates that free capture substance remained in the test sample and that the amount of detected substance in the original sample was no greater than a readily-determined threshold amount. The qualitative assay can readily be constructed to set a desired detection limit, as needed, by adjusting the known amount of capture substance used to make the test sample.

A device suitable for carrying out the methods of the invention provides a substantially electrically non-conductive base and two spaced-apart electrical conductors superposed on the base wherein the conductors define on the base a path between the conductors. The conductors can be connected to an electrical energy source to form an electrical circuit which includes the path. The “substantially electrically non-conductive” nature of the base is determined relative to the electrical conductivity of a metal layer that can form on the path during the assaying process. Preferably, the difference in electrical conductivity between the base material and the metal layer is 4 or 5 orders of magnitude for clear identification of a change in electrical conductivity on the electrical circuit. Exemplary devices include the devices described in U.S. Pat. Nos. 4,794,089, 5,137,827 and 5,284,748, and in published international patent application number PCT/US99/06145 (publication number WO 00/02047), each of which is incorporated herein by reference in its entirety.

The following Examples demonstrate the principles of the invention by comparing the detection sensitivity limits using HRP-streptavidin and traditional gold-streptavidin conjugates on a modified polymer surface and by using the conjugates in related methods for detecting a nucleic acid in a sandwich assay using a surface-bound DNA capture probe and a biotinylated probe for indirectly linking the HRP-streptavidin and gold-streptavidin conjugates to the detected nucleic acid. The examples are illustrative only and are not intended to limit the scope of the invention. Rather, the invention embraces all such modifications and variations as come within the scope of the accompanying claims.

### EXAMPLE 1

Polymer surfaces were coated with an amino acid copolymer, poly-phenylalanine-lysine. A linking group containing a biotin moiety was attached to the amine residues of the lysine. Serial dilutions of gold and horseradish peroxidase labeled streptavidin were prepared and then reacted with the immobilized biotin for a fixed period of time. After the appropriate chemical treatment steps the surface was subjected to silver enhancement and the conductivity of the resulting silver film was measured.

### Preparation of Poly-phe-lys Coated Polymer Sheets

Squares of polystyrene or polyester sheets were soaked for 30 minutes in ethanol and rinsed with deionized water two times. The squares were then soaked for 15 minutes in 2% Alconox and rinsed with deionized water twice. Following the two wash steps the squares were immersed in a solution of 0.1 mg/ml poly-phe-lys in 2 M NaCl, 50 mM KH₂PO₄ (pH 7.1) for 1 hour at 25° C. The squares were rinsed in 50 mM KH₂PO₄ (pH 7.1) and then rinsed in deionized water and stored at 4° C.

### Direct Biotinylation of Modified Polymer Surfaces

The biotinylation reagent was prepared by adding biotinamidocaproic acid 3-sulfo NHS ester to a 50 mM KH₂PO₄ (pH 7.1) at a level of 0.8 mg/ml. Designated locations on the poly-phe-lys coated polymer squares were spotted with 50 μl of the biotinylation reagent and incubated 2 hours at 25° C. and 100% relative humidity. The drops were aspirated by vacuum and then rinsed with deionized water.

### Binding of Streptavidin or Avidin Conjugates to Biotinylated Polymer Surfaces

Serial dilutions of gold and horseradish peroxidase streptavidin conjugates were prepared in phosphate buffered saline (PBS) with 1% BSA. Volumes of 10 μl were applied to specific locations of the biotinylated polymer surface. Streptavidin conjugates were incubated for 16 hours at 4° C. and 100% humidity. The drops were aspirated by vacuum and then rinsed with deionized water.

### Detection with Gold-streptavidin

Gold-streptavidin was directly detected by silver enhancement. The silver enhancing solution was prepared by combining 260 μl 43 mM AgNO₃, and 660 μl 90 mM hydroquinone/0.3 M citrate buffer pH 3.8. A 25 μl volume of enhancing solution was spotted on each designated location. The reaction was incubated 30 minutes in the dark and rinsed in deionized water.

Detection with HRP-streptavidin

The immobilized HRP streptavidin was detected by allowing the peroxidase to catalyze the deposition of a metal-polymer precipitate that could subsequently be silver enhanced to produce a conductive surface. A 10.5 mg tablet of diaminobenzidine (DAB) and 30 mg of urea hydrogen peroxide (UHP) were dissolved in 100 ml Bis-tris buffered nitrate (TBN)/10 mM NiCl₂ at pH 6.5 and filtered. The HRP-streptavidin labeled sheets were immersed in the DAB/UHP/Ni solutions for 5 minutes at 25° C. During this time a precipitate consisting of oxidized DAB and Ni was formed. The DAB/UHP/Ni solution was removed, and the sheets were rinsed with deionized water. The sheets were then soaked in a freshly prepared 10 μM AuCl₄ solution for 10 minutes and rinsed in deionized water. The silver enhancing solution was prepared by combining 260 μl 43 mM AgNO₃ and 660 μl 45 mM hydroquinone/0.15 M citrate buffer pH 3.8. A 25 μl volume of enhancing solution was spotted on each designated location. The reaction was incubated 15 minutes in the dark and rinsed in deionized water.

Comparison of Detection Sensitivity for Gold and HRP Labeled
Streptavidin

| Streptavidin | | |
|---|---|---|
| conjugate | Gold | HRP |
| Calculated density | Conductance | Conductance |
| Molecules/mm² | 1/Ω × 10⁶)/mm | 1/Ω × 10⁶)/mm |
| 3 * 10¹⁰ | 3200 | 480 |
| 1 * 10¹⁰ | 190 | 460 |
| 3 * 10⁹ | 0 | 520 |
| 1 * 10⁹ | 0 | 730 |
| 3 * 10⁸ | 0 | 480 |
| 1 * 10⁸ | 0 | 460 |
| 3 * 10⁷ | 0 | 100 |
| 1 * 10⁷ | 0 | 8 |
| 0 | 0 | 0 |

(Volume * conjugate concentration)/cross sectional drop area = cross sectional area density

### EXAMPLE 2

A surface-immobilized biotinylated nucleic acid bound to streptavidin-HRP was exposed at 37° C. for 5-15 minutes to 0.22 mg/ml 3,3′-DAB, 0.6 mg/ml urea hydrogen peroxide, 10 mM NiCl₂, 0.05 M Tris pH 7.6, 0.16 M NaCl, and 0.05% Tween. The surface was then washed using deionized water, exposed to a solution of 10 μM HAuCl₄ for 15 minutes at 25° C. and then washed again using deionized water. Then, the surface was exposed at 4-10° C. to 12 mM AgNO₃ and 65 mM hydroquinone in a standard silver development buffer (0.2 M citrate buffer, pH 3.7) for 15 minutes to produce a conductive metal layer. The surface was then washed again using deionized water. The resistance or conductivity of the metal layer was measured by providing a pair of spaced apart electrical conductors attached to an ohmmeter or a conductivity meter.

Applicant observed that where HRP was bound on the surface between the conductors in varying amount, it was possible to quantitatively distinguish the amounts of HRP by measuring the resistance or conductivity of the conductive layer.

### EXAMPLE 3

An amine labeled capture DNA probe was immobilized on a poly-phe-lys coated polymer surface by means of a bifunctional coupling reagent. Additional sites capable of binding nonspecifically to the detector probe were then reacted with a blocking reagent. Serial dilutions of a biotinylated DNA detector probe were hybridized to the capture probe for a fixed period of time. The biotinylated probe was then detected by means of gold or HRP streptavidin conjugates.

### Immobilization of DNA Capture Probes on Substrate Surfaces

The polymer surface was coated with poly-phe-lys as described in Example 1. A 5.7 mg aliquot of bis(sulfosuccinimidyl) suberate (BS³) was dissolved in 100 μl 0.1 M KH₂PO₄ pH 7.1. A 5 μl volume of the BS³ solution was combined with 5 μl of an amino-labeled oligonucleotide stock at 250 μM. The reaction was incubated for 10 minutes and then added to 990 μl of 0.1 M KH₂PO₄ pH 7.1. Unreacted BS³ was removed by size exclusion chromatography. Designated locations on the poly-phe-lys coated polymer sheets were spotted with 20 μl aliquots of the BS³ oligo-solution and incubated 1 hour at 25° C. in 100% relative humidity. Sheets were washed in deionized water. Polystyrene sheets were soaked for one hour in 0.2 M NaOH/0.1% SDS to remove capture probe that was not covalently bound. Melinex (polyester) sheets were soaked for one hour in 0.1 M KH₂PO₄ pH 7.1/0.1% Tween. The sheets were rinsed in 50 mM KH₂PO₄ (pH 7.1) and then deionized water.

### Post Immobilization Blocking of the Substrate Surface

A 3.8 mg aliquot of disuccinimidyl suberate (DSS) was dissolved in 3 ml of DMSO. The DSS solution was diluted to 15 ml with 0.1 M KH₂PO₄ pH 7.1. The polymer sheets were incubated one hour in the DSS solution. The sheets were rinsed in deionized water and stored at 4° C.

### Hybridization of DNA Detector Probes

Hybridization solutions were comprised of serial dilutions of biotinylated detector probe in hybridization buffer. The composition of the hybridization buffer was 1 M NaNO₃, 20 mM Tris pH 8, 1 mM EDTA, and 0.01% SDS. Hybridization reactions were carried out for 16 hours at 56° C.

### Detection with Gold-streptavidin

The gold-streptavidin was bound to the hybridized biotinylated probe and was silver enhanced. Gold-streptavidin solution was prepared by diluting the commercial stock to a concentration of 10 ng/μl in nitrate buffer (0.15 M NaNO₃, 50 mM tris (pH 8.0) and 1% BSA). Volumes of 25 μl were placed on the hybridized spots and incubated for 16 hours at 4° C. and 100% humidity. The drops were aspirated by vacuum and then rinsed with nitrate buffer. Silver enhancement was carried out using the same solutions and conditions as those described in Example 1 for gold-streptavidin.

### Detection with HRP-streptavidin

The HRP-streptavidin was bound to the hybridized biotinylated probe. The immobilized HRP catalyzed the deposition of a polymer-metal precipitate. The precipitate was treated with a gold chloride toning solution followed by silver enhancement. The HRP-streptavidin solution was prepared by diluting the commercial stock to a concentration of 100 pg/μl in hybridization buffer with 1% BSA. Volumes of 25 μl were placed on the hybridized spots and incubated for 16 hours at 4° C. and 100% humidity. The drops were aspirated by vacuum and then rinsed with hybridization buffer. A 3.5 mg tablet of diaminobenzidine (DAB) and 30 mg urea hydrogen peroxide (UHP) were dissolved in 100 ml Bis-tris buffered nitrate (TBN)/10 mM NiCl₂ at pH 6.5 and filtered. The HRP-streptavidin labeled sheets were immersed in the DAB/UHP/Ni solution for 5 minutes at 25°. The DAB/UHP/Ni solution was removed and the sheets were rinsed with deionized water. The sheets were soaked in freshly prepared 10 μM HAuCl₄ for 10 minutes and then washed in deionized water. Silver enhancement was carried out using the same solutions and conditions as those described in Example 1 for HRP-streptavidin.

The observed sensitivities are noted in the following tables which demonstrate that the detection limit is enhanced by at least two orders of magnitude using HRP-Streptavidin labeling in accordance with the invention.

Sensitivity of Biotinylated DNA Probe Detection
on Polystyrene (PS) and Melinex (Mel) Resistor Sheets
Using Gold-Streptavidin Labeling

| Probe | | |
|---|---|---|
| Concentration | Polystyrene | Melinex |
| M | (1/Ω × 10⁶)/mm | (1/Ω × 10⁶)/mm |
| 10⁻⁷ | 75 | 150 |
| 10⁻⁸ | 75 | 140 |
| 10⁻⁹ | 70 | 140 |
| 10⁻¹⁰ | 10 | 45 |
| 10⁻¹¹ | 0 (visual +) | 0 (visual +) |
| 10⁻¹² | 0 | 0 |

Sensitivity of Biotinylated DNA Probe Detection
on Polystyrene (PS) and Melinex (Mel) Resistor Sheets
Using HRP-Streptavidin Labeling

| Probe | | |
|---|---|---|
| Concentration | Polystyrene | Melinex |
| M | (1/Ω × 10⁶)/mm | (1/Ω × 10⁶)/mm |
| 10⁻⁷ | 240 | 120 |
| 10⁻⁸ | 230 | 75 |
| 10⁻⁹ | 160 | 90 |
| 10⁻¹⁰ | 120 | 90 |
| 10⁻¹¹ | 150 | 55 |
| 10⁻¹² | 100 | 75 |
| 10⁻¹³ | 15 | 5 |
| 10⁻¹⁴ | 0, (visual +) | 0, (visual +) |

A visual positive indicates that sufficient silver was deposited to produce a discernable spot. However, the silver was deposition was not complete enough to yield a conductive surface.

I claim:

1. A method for detecting a detected substance in a sample, the method comprising the steps of:
providing, in a path between spaced-apart electrical conductors on a substantially non-conductive base, aggregates of a complex between the detected substance, a capture substance specifically bound thereto and an oxidative enzyme;
exposing the aggregates in the path to an substrate capable of being oxidized by the oxidative enzyme and to transition metal ions so that the substrate is oxidized and the metal ions are reduced to zero-valence metal atoms that precipitate on the aggregates to form a transition metal layer; and
measuring electrical resistance of an electrical circuit comprising the conductors, the transition metal layer and an electrical energy source, whereby a change in resistance relative to a control lacking the detected substance indicates the detected substance in the sample.

2. The method of claim 1 further comprising the step of depositing a second metal layer on the transition metal precipitate layer, the second metal layer being more catalytic for reducing silver ions than the transition metal layer.

3. The method of claim 2, wherein the step of depositing the second metal layer comprises the step of exposing the transition metal precipitate layer to a metal salt under suitable metal depositing conditions, the metal being selected from the group consisting of gold, platinum and palladium.

4. The method of claim 2 further comprising the step of depositing silver metal on the second metal layer.

5. The method of claim 4, wherein the step of depositing the silver metal comprises the step of exposing the second metal layer to a silver salt and a reducing agent.

6. The method of claim 5, wherein the silver salt is selected from the group consisting of silver nitrate, silver lactate and silver acetate.

7. The method of claim 1, wherein the oxidative enzyme is selected from the group consisting of a peroxidase and an acetate.

8. The method of claim 1, wherein the enzyme is horseradish peroxidase.

9. The method of claim 1, wherein the substrate capable of being oxidized by the oxidative enzyme is selected from the group consisting of diaminobenzidine and 3,3′,5,5′-tetramethylbenzidine.

10. The method of claim 1, wherein the oxidative substrate is diaminobenzidine.

11. The method of claim 1, wherein the transition metal is selected from the group consisting of nickel, cobalt, copper, lead, and cadmium.

12. The method of claim 1, wherein the detected substance is selected from the group consisting of a nucleic acid and a polypeptide.

13. The method of claim 1 wherein the detected substance is a nucleic acid labeled with biotin and the capture substance is a nucleic acid anchored to the path that hybridizes at least in part to the detected biotin-labeled nucleic acid, wherein the oxidative enzyme is linked to the complex via a biotin-streptavidin linkage.

14. The method of claim 1 wherein the detected substance is a nucleic acid, the capture substance is a nucleic acid anchored to the path that hybridizes at least in part to the detected nucleic acid, the complex further comprising a biotin-labeled nucleic acid hybridized to the detected nucleic acid, wherein the oxidative enzyme is linked to the complex via a biotin-streptavidin linkage.

15. The method of claim 1 wherein the detected substance is an antigen, the capture substance is anchored to the path and is a first antibody capable of specific binding to the antigen, wherein the oxidative enzyme is linked to the complex via a second antibody capable of specific binding to the antigen.
